(19) United States
(12) Patent Application Publication
(10) Pub. No.: US 2003/0065285 A1
(43) Pub. Date: Apr. 3, 2003

(54) Method and apparatus for increasing flux during reverse iontophoresis

(76) Inventor: William I. Higuchi, Salt Lake City, UT (US)

Correspondence Address: REED & EBERLE LLP, 800 MENLO AVENUE, SUITE 210, MENLO PARK, CA, 94025, US

(21) Appl. No.: 09/911,594

(22) Filed: Jul. 23, 2001

(51) Int. Cl. A61N 1/30

(52) U.S. Cl. 604/20

(57) ABSTRACT

The present invention provides a method and device that substantially increases reverse iontophoretic flux and therefore, noninvasive extraction of uncharged and charged permeant molecules alike through the skin. By substituting the mobile co-ions, which are capable of easily entering the pores from the receiver compartment of a reverse iontophoretic extraction device with large conductive polyelectrolytes within the reservoir that do not appreciably enter the pores, the invention significantly improves the amount of analyte extracted, improves device performance, decreases energy requirements, increases battery life, reduces the potential for irritation, and improves accuracy, reproducibility, and precision.

### TECHNICAL FIELD

This invention relates generally to the use of iontophoresis for permeant transport and, more specifically, to a novel method for increasing the extraction of charged and uncharged permeants alike from the human body through a body surface and into a collection medium. This invention finds utility in any instance wherein a compound is removed from the body via iontophoresis, such as glucose monitoring, phenylalanine monitoring, therapeutic drug monitoring, fertility monitoring, monitoring for illicit drug use, noninvasive pharmacokinetic or toxicokinetic monitoring, and monitoring of any other body component, endogenous or introduced, that is a marker of health or disease. In addition, this invention may also reduce the changes in flux encountered during iontophoresis as well as reduce intersubject variability.

### BACKGROUND

Iontophoresis is the process of using a low level electrical current to evince the movement of permeant molecules or ions across a body surface. Most scientists believe that iontophoretic transport occurs within aqueous pores either previously present in the skin structure or through pores created by the electrical current, a phenomenon known as electroporation.

Reverse iontophoresis refers to the use of a mild electrical current to withdraw compounds from the body of a patient. The compounds can be withdrawn across any body surface, although the skin is chosen most often because of its large surface area and easy accessibility. Reverse iontophoresis can be used to withdraw both charged and uncharged compounds from the body.

Noninvasive analyte extraction from the body can take on many different forms. One can use reverse iontophoresis to extract glucose from the body and correlate the extracted glucose concentration with blood glucose concentration for noninvasive blood glucose monitoring, thereby providing a complete picture of an individual's blood glucose profile on a real-time basis. In addition, one can use reverse iontophoresis to extract phenylalanine from the body of a patient with phenylketonuria to measure blood levels of phenylalanine and detect toxic accumulation of phenylalanine in the patient's blood or as a screening method to non-invasively identify patients with elevated phenylalanine levels. Another use of reverse iontophoresis is to non-invasively extract and monitor narrow therapeutic window agents, such as amino glycoside antibiotics, antiepileptics, cardiac glycosides, or anticoagulants, to adjust dosing and ensure a therapeutic effect, yet avoid toxicity. Still other uses for non-invasive reverse iontophoresis are to detect the presence of illicit drugs or other toxic substances in the body, as well as to non-invasively perform toxicokinetic or pharmacokinetic monitoring.

Systems for transporting ionized substances through the skin have been known for decades. British Patent Specification No. 410,009 (1934) describes an iontophoretic delivery device that overcame one of the disadvantages of earlier such devices, specifically, the need for a patient to be immobilized near a source of electric current. This device was made by forming a galvanic cell, which itself produced the current necessary for iontophoretic delivery from the electrodes and the material containing the drug to be delivered. Unlike previous iontophoretic delivery systems, the device allowed the patient to move around during drug delivery and thus minimized interference with the patient's daily activities.

In modem iontophoretic devices, at least two electrodes are used. Each of these electrodes is positioned so as to be in intimate electrical contact with some area of the body surface, i.e., skin or mucosal tissue. In iontophoretic drug delivery, one electrode, called the active or donor electrode, is the electrode from which the drug is delivered into the body. The other electrode, called the counter or return electrode, serves to close the electrical circuit through the body. If the ionic substance to be driven into the body is positively charged, then the positive electrode (the anode) will be the active electrode and the negative electrode (the cathode) will serve as the counter electrode, thereby completing the circuit. Conversely, if the ionic substance to be delivered is negatively charged, then the cathode will be the active electrode and the anode will be the counter electrode.

In analyte extraction, the electrode that receives the analyte from the body can be termed the receiver or sensing electrode, while the second electrode can be termed the indifferent, or return, electrode. If the substance being extracted from the body is a cation (positively charged), then the cathode will function as the receiver electrode. Conversely, if the extracted substance is an anion, the anode will serve as the receiver electrode. If the extracted substance is uncharged, however, the anode or cathode can function as the receiver electrode; although the cathode will most likely be the receiver electrode, due to the characteristics of electroosmotic flux, which flows from anode to cathode under physiological conditions.

In conjunction with the patient's skin, the circuit is completed by connection of the electrodes to a source of electrical energy, e.g., a battery, and usually to circuitry capable of controlling the amount of current passing through the device.

Iontophoretic analyte extraction devices usually include a reservoir for collection of the analyte. Examples of such reservoirs or sources include: a pouch, as described in Jacobsen, U.S. Pat. No. 4,250,878; a pre-formed gel body, as disclosed in Webster, U.S. Pat. No. 4,382,529 and Ariura et al. U.S. Pat. No. 4,474,570; a receptacle containing a liquid solution, as disclosed in Sanderson, et al., U.S. Pat. No. 4,722,726; a wettable woven or non-woven fabric; a sponge material; or any combination thereof. Such reservoirs are connected to the anode or the cathode of an iontophoretic device to provide a collection point for one or more desired agents.

In iontophoretic systems, and particularly in reverse iontophoretic systems, electroosmosis is typically dependent upon sodium ion flow into the cathode from the body. Electroosmotic flow is created by an electrical volume force that is a result of mobile counter-ions in pores acting on the solvent. When co-ions are present in the receiving chamber of a reverse iontophoretic electrode, they also impart an electrical volume force in the opposite direction, albeit somewhat less efficiently than the convection imparted by the sodium ion, due to its generally lower concentration in the negatively charged pores, thus impeding the convectional flow. Conventional reverse iontophoretic devices contain a high concentration of small, highly mobile co-ions in the receiver chamber. As current is applied, the co-ions enter the transport pathways and create an inward driving force that impedes the outward extraction convection force.

In order to optimize reverse iontophoretic methods and devices, it is necessary to develop reproducible extraction processes and to increase the rate of analyte extraction. Various methods have been explored to increase the rate of electroosmotic extraction. Santi and Guy in Santi et al. (1996), J. Cont. Rel. 38:159-165 showed that the rate of electroosmotic flux could be increased by lowing the electrolyte ionic concentration in both the anode and cathode chamber. The method disclosed by Santi et al. presents many potential disadvantages. use of low ionic strength solutions in the extraction compartment has many shortfalls. The extremely low ionic strength solutions used in the method of Santi et al. may 1) raise the voltage required to drive the electrical current across the skin, thereby increasing the potential for skin irritation and 2) provide an inadequate number of ions to support the electrochemistry Ag/AgCl couple. Further, although using low and seemingly impractical solution ionic strengths, the Santi and Guy were able to realize a maximum improvement in electroosmotic flux of only about two-fold into the cathode chamber and even less into the anode chamber. The novelty of the current invention lies in the fact that despite using solutions with approximately a 10-fold higher ionic strength than previous researchers, the current invention will achieve enhancements in electroosmotic flux many times greater than that observed by Santi and Guy.

Santi and Guy, in Santi et al. (1996), J. Cont. Rel. 42:29-36, further demonstrated that the use of divalent ions in the anode chamber increased electroosmotic flow toward the anode and other formulation in the cathode increased electroosmotic flow towards the cathode, seemingly by affecting the shielding of charged groups within the pores. The compounds these researchers used, heparin, calcein, and EDTA, are impractical to use in a commercial iontophoretic device. As the compounds used by Santi and Guy can easily enter the pore pathways during iontophoresis, their toxicity and ability to induce irritation are is high. Also, as heparin, calcein, and EDTA readily enter the pores, regulatory approval of a device containing these compounds is sure to be lengthy and fraught with difficulty.

The present invention substantially increases electroosmotic solvent flow and therefore, noninvasive extraction of uncharged permeant molecules through the skin. By replacing the mobile co-ions, which are capable of easily entering the pores from the receiver compartment of a reverse iontophoretic extraction device with large conductive polyelectrolytes within the reservoir that do not appreciably enter the pores, the invention significantly improves the amount of analyte extracted, improves device performance, decreases energy requirements, increases battery life, reduces the potential for irritation, and improves accuracy, reproducibility, and precision.

While polyelectrolytes have been employed in known iontophoretic devices, no such device has incorporated polyelectrolytes in reverse iontophoretic applications. U.S. Pat. No. 5,882,677 to Kupperblatt discusses a hydrogel reservoir containing a water-soluble polyelectrolyte and a fluid for use in two-compartment iontophoretic patches. The polyelectrolyte is used as an ion exchange resin to control Ag⁺ migration resulting from the oxidation of silver metal at the anode. However, the use of polyelectrolytes to enhance electroosmotic flux was an unexpected observation and is unobvious to one skilled in the art. Thus, the present invention is novel and presents clear advantages over currently used reverse iontophoretic devices and methods.

### SUMMARY OF THE INVENTION

In one main aspect of the current invention, a device is provided that increases analyte flux during reverse iontophoresis conducted on a region of body tissue comprising (i) a first electrode assembly adapted for placement in analyte receiving relation with the body tissue comprising a reservoir for containing an analyte extracted from the body and one polyelectrolyte or multiple polyelectrolytes; (ii) a second electrode assembly adapted to be placed in ion transmitting relation with the body tissue at a location spaced apart from the first electrode assembly; and (iii) an electrical current source, electrically connected to the first and second electrode assemblies.

In another aspect of the invention, a method for extracting an analyte across a region of body tissue is provided. A first electrode assembly is placed in contact with a body tissue, consisting of an electrically conducting medium comprising a polyelectrolyte composition that cannot readily pass into the body tissue when an electrical current is applied. Next, a second electrode assembly, placed in an ion transmitting relation with the body surface, is positioned in contact with the body tissue at a location spaced apart from the first electrode assembly. Finally, an electrical current is applied across the region of body tissue via the first and second electrode assemblies. The applied current is of a magnitude, voltage and duration effective to induce electroosmosis and transport the analyte to the first electrode assembly.

Preferably, the body tissue to which an electrical current is applied is skin or mucosal tissue. The applied current may be either direct or alternating, or a mixture of the two, and the extracted analyte may be glucose, phenylalanine, or a marker of a specific disease, condition, or chemical, either endogenous or exogenous in nature, either charged or uncharged. If desired, more than one analyte may be extracted at a time at the same or at both electrodes.

In a third aspect of the invention, an improved method for extracting an analyte from a region of body tissue, comprising (a) placing a first electrode assembly and a second electrode assembly on an individual's body surface in ion-transmitting relation thereto, the first and second electrode assemblies spaces apart at a selected distance, and (b) applying an electrical current across the region of body tissue via the first and second electrode assemblies, with a voltage and duration effective to induce electroosmosis and transport the analyte to the first electrode assembly at a transport rate having a mean steady state permeability that varies when the method is applied to different regions of body tissue, the improvement comprising incorporating a polyelectrolyte composition into the first electrode assembly that cannot readily pass into the body tissue when an electrical current is applied, said polyelectrolyte composition effective to provide a substantial decrease in the variability the mean steady state permeability when the method is applied to different regions of body tissue. The decrease in variability of the mean steady state permeability is preferably at least about 30%, more preferably at least about 50%, and most preferably at least about 90% of the variability observed when the polyelectrolyte is not present.

### BRIEF DESCRIPTION OF THE DRAWINGS

FIG. 1 presents a schematic diagram of conventional electroosmotic transport. The direct electrostatic forces of the main cation in the body, the sodium ion (Na⁺), cause it to be transported from the anode to the cathode. Conversely, anions, also known as co-ions, (X⁻, mainly Cl⁻) move from the cathode to the anode. The Net Convection Vector is the difference between the Anode→Cathode Convective Vector due to solvent flow towards the cathode (in this case caused by Na⁺ ion flux) and the Cathode→Anode Convection Vector due to flow of the co-ions towards the anode (evinced by X⁻ or Cl⁻ ion flux).

FIG. 2 presents a schematic diagram of electroosmotic transport using the method of the invention. Although the Anode→Cathode Convection Vector is unchanged with respect to FIG. 1, the dearth of highly mobile co-ions causes a large increase in the Net Convection Vector as the Cathode→Anode Convention Vector is minimized.

FIG. 3 present a schematic diagram of the experimental apparatus used to test the invention.

### DETAILED DESCRIPTION OF THE INVENTION

Definitions and Overview:

Before describing the present invention in detail, it is to be understood that this invention is not limited to specific drug delivery systems, reverse iontophoresis extraction systems, device structures, enhancers, polyelectrolytes, or carriers, as such may vary. It is also to be understood that the terminology used herein is for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to “a drug” includes a mixture of two or more drugs, reference to “a co-ion” includes one or more co-ions, reference to “an analyte” includes one or more analytes, and the like.

In describing and claiming the present invention, the following terminology will be used in accordance with the definitions set out below.

Herein the terms “iontophoresis” and “iontophoretic” are used to refer to the transdermal delivery of pharmaceutically active agents by means of an applied electromotive force to an agent-containing reservoir. The terms “iontophoresis” and “iontophoretic” are also meant to refer to “reverse iontophoresis,” “reverse iontophoretic,” “electroosmosis,” and “iontohydrokinetic” or “iontohydrokinetic.” The terms “reverse iontophoresis,” “reverse iontophoretic,” and “analyte extraction” are used to refer to the collection of analytes from the body by means of an applied electromotive force to an analyte-collecting reservoir.

The terms “current” or “electrical current,” when used to refer to the conductance of electricity by movement of charged particles, are not limited to “direct electrical current,” “direct current,” or “constant current.” The terms “current” or “electrical current” should also be interpreted to include “alternating current,” “alternating electrical current,” “alternating current with direct current offset,” “pulsed alternating current,” and “pulsed direct current.”

During iontophoresis, certain modifications or alterations of the skin occur, for example, changes in permeability, due to mechanisms such as the formation of transiently existing pores in the skin, also referred to as “electroporation.” Any electrically assisted transport of species enhanced by modifications or alterations to the body surface (e.g., formation of pores in the skin and “electroporation”) are also included in the term “electrotransport” as used herein. Thus, as used herein, the terms “electrotransport,” “iontophoresis,” and “iontophoretic,” further refer to the transport of permeants by the application of an electric field regardless of the mechanisms.

The term “pore” is used to describe any transport pathway through the tissue, whether endogenous to the tissue or formed by electroporation.

The term “polyelectrolyte” is used to describe any molecule with two or more charged group and associated co-ions. The term “polyelectrolyte” also includes a mixture or mixtures of different “polyelectrolytes” or similar “polyelectrolytes” with different molecular weight distributions. The “polyelectrolyte” may be a single molecule or an aggregate of molecules, such as micelles (both cationic and anionic) and liposomes (again both cationic and anionic). A “polyelectrolyte”, as used in this invention, should be regarded as a molecule or aggregate of molecules with a significantly high molecular size as to have impeded transport into or through pores. The terms “polyelectrolyte” and “polyelectrolyte composition” are equivalent with respect to this invention.

The term “co-ion” is used to define an ion that is transported in the same direction as the active agent (in the case of drug delivery), or transported in the same direction as the permeant extracted from the body. Other terms that are synonymous with “co-ion” are “background ion,” “background electrolyte,” and “excipient ion”.

The terms “body surface” and “tissue” are used to refer to skin or mucosal tissue, including the interior surface of body cavities that have a mucosal lining. The term “skin” should be interpreted as including “mucosal tissue” and vice versa.

A “region” of a tissue refers to the area or section of a tissue that is electroporated via the application of one or more electrical signals and through which an agent is transported. Thus, a region of a body surface refers to an area of skin or mucosal tissue through which an active agent is delivered or an analyte is extracted.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. The term “treatment” is also used to refer to the extraction of a substance through a tissue for the purpose of analytical quantitation or qualification.

The terms “pharmacologically active agent,” “active agent,” “pharmaceutical agent,” “pharmaceutically active agent,” “drug,” and “therapeutic agent,” are used interchangeably herein to refer to a chemical material or compound suitable for delivery across a tissue (e.g., transdermal or transmucosal administration), which induces a specific desired effect. The terms include agents that are therapeutically effective as well as those that are prophylactically effective. Also included are derivatives and analogs of those compounds or classes of compounds specifically mentioned above, including active metabolites of the therapeutic agent, which induce the desired effect.

All of the descriptions contained herein should not be limited to constant current or direct current methods. All descriptions should also be interpreted to include alternating current or alternating current with direct current offset.

Iontophoretic transport occurs in three basic manners: direct electric field effect, electroosmosis, and electroporation. It is known that during direct current (DC) iontophoresis, the applied current causes an enlargement of pre-existing skin pores or causes pores in the skin to form (electroporation) and enlarge resulting in reduced electrical resistance. In addition, the direct current changes the net charge density of the pores. See, for example, U.S. Pat. No. 5,374,242 to Haak et al. and U.S. Pat. No. 5,019,034 to Weaver et al. Electroporation does not itself affect permeant transport but merely prepares the tissue thereby treated for permeant transport by any of a number of techniques, one of which is iontophoresis. The method of the invention serves to enhance the effects of electroosmosis and is not dependent on the occurrence of electroporation.

The following discussion attempts to explain the theory behind the electric field effect and electroosmosis. The discussion is illustrative only and should not be considered limiting, as other aspects, such as polyelectrolyte entering the pores, may also explain the observed phenomenon.

Electroosmotic flow is bulk fluid flow that occurs when a voltage difference is imposed across a charged membrane. Electroosmotic flow occurs in a wide variety of membranes and is usually in the same direction as the flow of counter-ions for analyte extraction and is most often in the same direction of co-ion flow for drug delivery. Since most mammalian tissues have a net negative charge at physiological pH values, counter-ions are positive ions and electroosmotic flow occurs from anode to cathode. Water carried by ions as ‘hydration water’ does not contribute significantly to electroosmotic flow. Rather electroosmotic flow is caused by an electrical volume force acting on the mobile counter-ions. See, Pikal M J (2001) “The Role of Electroosmotic Flow in Transdermal Iontophoresis,” Adv Drug Deliv Rev, 46:281-305.

The speed of an ion as it moves under the influence of an electric field is called its mobility, μ; i.e., μ=νE⁻¹, where ν is velocity of the ion and E is the applied potential gradient. The Debye-Huckel theory accounts for many of the phenomena observed in dilute solutions of strong electrolytes. The three basic assumptions in the Debye-Huckel theory are that:

1. Strong electrolytes are completely dissociated into ions;

2. Deviations from ideal behavior result from electrostatic attractions between the charges of the ions; and

3. A given ion will have more ions of the opposite charge close to it than ions of the same charge; this cluster of ions is called the ionic atmosphere.

Two effects prevent the ions from moving at their maximum expected speed:

1. The relaxation effect (also called the asymmetry effect) occurs because the central ion tries to move out of its ionic atmosphere. The symmetry present before application of the electrical potential is distorted in such a way that an unbalanced force acts on the central ion, tending to hold it back.

2. The electrophoretic effect occurs because the atmosphere and the central ion are pulled in opposite directions. The ions are usually solvated, so solvent molecules are pulled along. Again, the central ion is held back by the flow of the solvent against which it is trying to move.

The assumptions of the Debye-Huckel theory, and in particular assumption 2, explain deviations from ideal or maximum velocity of an ion predicted in an aqueous filled pore. When an electrical field is imposed, both positively and negatively charged ions move in the direction of their respective electrostatic gradient: the anions towards the anode, cations towards the cathode. When the electrolyte is NaCl, both Na⁺ and Cl⁻ ions are present in the transport pathways. If both of these ions are present in uncharged aqueous pores at the same concentration, there is no net convective transfer of water in either direction: the forward and backward forces cancel each other out. However, because the pores are negatively charged at physiological pH, (e.g. a stratum corneum pore at physiological pH) Na⁺ has a higher concentration than chloride in the pores. Therefore, there is a net convection of water in the direction towards the cathode. The force imparted by the higher concentration of Na⁺ ions and the resulting water convection will further impart a convective force on all ions or soluble molecules in solution and cause their movement.

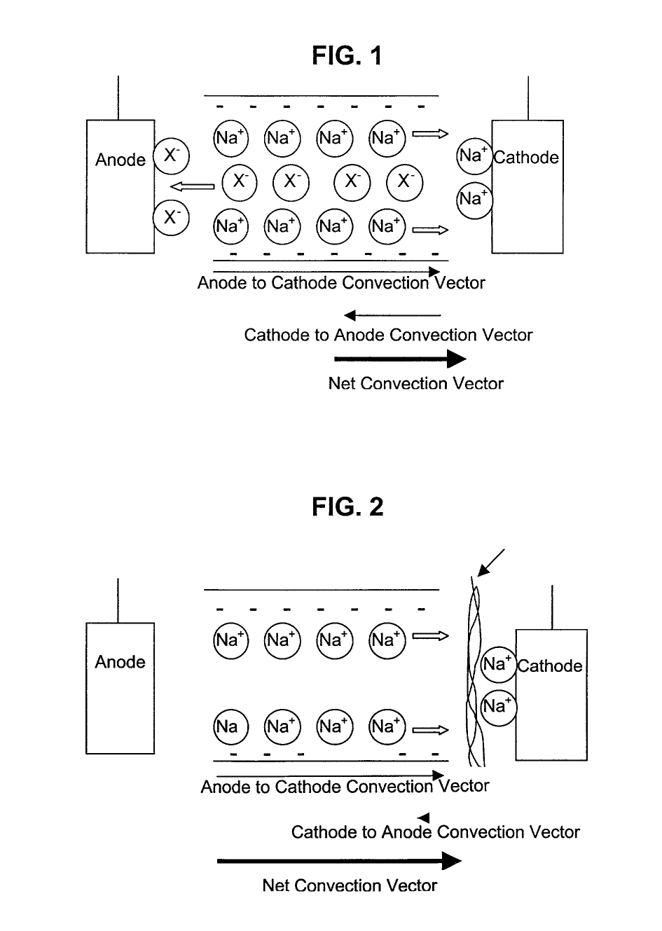

The conventional electroosmosis theory explains why the efficiency of electroosmotic flux of charged and uncharged molecules is always less than predicted. The force imparted by the counter ion, Cl⁻, on the water convection substantially impedes and counteracts the convection imparted by the force of the moving Na⁺ ion and vice-versa. This phenomenon is depicted by FIGS. 1 and 2. In FIG. 1, the sodium ion creates a forward convection vector in the direction of anode to cathode and the co-ion (X⁻ or chloride) creates a reverse convection vector in the direction of cathode to anode. The net convection vector can be represented by the difference between the two vectors. However, when the co-ion is removed from the collecting electrode, as shown in FIG. 2, there is no reverse convection component, and therefore the forward convection vector imparted by the sodium ion is allowed to proceed unimpeded. FIGS. 1 and 2 are illustrative and should not be considered to be limiting. As will be discussed subsequently, this invention can also aid in the electroosmotic flow in the direction of cathode to anode. In such a case, the convection vector direction in 1 and 2 will change and the Na⁺ will become X⁻ and vice versa.

As with most analyte extraction from the body with reverse iontophoresis, the net solvent convective flow and resulting analyte movement is in the direction of anode to cathode. A substance that can provide electrical conduction in the cathode, with minimal transport into and through the pores and while not imparting solvent convective force contrary to the desired permeant flux, should greatly improve the movement of uncharged molecules or ions through the body surface.

Although most uses of electroosmosis utilize net convection in the direction of anode to cathode, this invention is not limited to transport in the direction of anode to cathode. A polycationic substance that provides for electrical conduction in the anode, with minimal transport into and through the pores, will allow for an increased contribution of Cl⁻ towards the electroosmotic flux and will increase permeant transport in the direction of cathode to anode. Unexpected advantages of the reversal of electroosmotic flow could include a possible decrease in irritation, decrease in electrical requirement, or increase in the amount of permeant extracted through the skin per unit time and increased precision, reproducibility, and accuracy.

This invention proposes using high molecular weight, charged polyelectrolyte polymers to provide an electrically conducting medium in the receiving electrode that will maximize iontohydrokinetic or electroosmotic flow during reverse iontophoresis. Such enhancements in the solvent flow may result in a 2 to 50 fold or more improvement in the reverse iontophoretic transport of permeants through the skin.

This invention is not limited to uncharged species as electroosmosis also increases the transport of many charged species. Nor is this invention limited to species whose transport occurs mainly or exclusively by electroosmosis. By eliminating the ionic environment and its influence on ionic movement of the oppositely charged species, the movement of all counter-ions, and not just Na⁺ and Cl⁻, will be enhanced. In a similar manner, this invention should not be limited to the extraction of uncharged species towards the cathode. Similar principles apply for extraction in the direction of the anode. By placing a polyanion, such as polystyrene sulfonate, in the cathode, or a polycation, such as DEAE-dextran in the anode, convective solvent flow, or direct electrostatic movement towards those respective chambers will be significantly enhanced.

The polyelectrolyte selected should have a molecular weight of about 1,000 or greater. Polyelectrolytes with strongly ionic groups such as sulfonates, carboxylates, phosphates, and quaternary ammonium groups may be used. Examples of materials useful as a backbone for the polyelectrolyte include dextrans, agarose, cellulose, and polystyrene, among others.

Examples of polyelectrolytes useful in this invention include, but are not limited to: cholestyramine, dextran carbonates, dextran sulfates, aminated styrenes, polyvinylimine, polyethyleneimine, poly(vinyl 4-alkylpyridinium), poly(vinylbenzyltrimethyl ammonium), polystyrene sulfonate, polymethacrylates, hyaluronate, alginate, acrylarnideo methyl propane sulfonates (poly-AMPS), hydroxyl ethyl methacrylates (poly-HEMA), and sodium polystyrene sodium sulfonate, DEAE Sephadex, QAE Sephadex, DEAE Sepharose, poly(N-tris[hydroxymethyl]methyl methacrylamide, DEAE trisacryl m, Q Sepharose, DEAE Sephacel, DEAD cellulose, epichlorohydrin triethanolamine cellulose, QAE cellulose, Amberject 4400, Dowex G-55, CM Sephadex, SO Sephadex, CM Sepharose, SP Sepharose, SP-Trisacryl Plus-M, SP-trisacryl M, CM cellulose, cellulose phosphate, sulfoxyethyl cellulose, Amberlite strongly acidic, Diaion Strongly acidic, Dowex-50W, Dowex 650C, Dowex G-26, Amberlite IRN-150, Amberlite MB-150, Dowex MR-3, Dowex MR-3C, benzoylated naphthoylated DEAE cellulose, benzyl DEAE cellulose, TEAE cellulose, Toyopearl DEAE-650C, Toyopearl DEAE 650-M, oxycellulose, Amberlite IRA-743, Amberlite IRA-900, Amberlite IRA-400, Amberlite IRA-402, Amberlite IRA-410, Amberlite IRA-420C, Amberlite A 5836, Amberlite IRA-458, Amberlite 16766, Dowex 1X2-100, Dowex 1X2-200, Dowex 1X2-400, Dowex 1X4-50, Dowex 1X4-100, Dowex 1X4-200, Dowex 1X4-400, Dowex 1X8-50, Dowex I9880, Dowex I0131, Dowex 1X8-100, Dowex 1X8-200, Dowex 1X8-400, Dowex 2X8-100, Dowex 2X8-200, Dowex 2X8-400, Diaion 1-3501, Diaion 1-3513, Diaion 1-3505, Diaion 1-3521, Diaion 1-3525, Diaion 1-3529, Diaion 1-3533, Amberlite IRA-92, Amberlite IRA-95, Amberlite IRA-96, Amberlite IRA-67, Dowex D2533, Dowex D3303, Dowex D5052, Diaion 1-3541, Duolite 1-0348, Amberlite 200, Amberlite IR-118H, Amberlite IR-120Plus, Amberlite IR-122, Amberlite IR-130C, Amberlite I 6641, Amberlite IRP-69, Dowex 50X1-100, Dowex 50X1-200, Dowex 50X1-400, Dowex 50X2-100, Dowex 50X2, 200, Dowex 50X2-400, Dowex 50X4-100, Dowex 50X4-200, Dowex 50X4-400, Dowex 50X4-200R, Dowex I 8880, Dowex 50X8-100, Dowex 50X8-200, Dowex 50X8-400, Diaion 1-3561, Diaion 1-3565, Diaion 1-3570, Diaion 1-3573, Diaion 1-3577, Diaion 1-3581, Duolite D 5427, Duolite D 5552, Amberlite DP-1, Amberlite IRC-50, Amberlite CG-50, Amberlite IRP-64, -Amberlite IRP-88, Amberlite D 7416, Diaion 1-3585, Diaion 1-3589, Diaion 1-3593, Duolite D 7416, Duolite D 5677, poly(acrylic acid-do-ethylene) sodium, sodium polyacrylate, poly(4-tert-butylphenol-co-ethylene oxide-co-formaldehyde) phosphate, poly (2-DEAE methacrylate) phosphate, poly(ethyl acrylate-co-maleic anhydride-co-vinyl acetate) sodium, polyethyleneaminosteramide ethyl sulfate, chlorosulfonated polyethylene, poly(ethylene-co-isobutyl acrylate-co-methacrylate) potassium, poly(ethylene-co-isobutyl acrylate-co-methacrylate) sodium, poly(ethylene-co-isobutyl acrylate-co-methacrylate) sodium zinc, poly (ethylene-co-isobutyl acrylate-co-methacrylate) zinc, poly(ethylene-co-methacrylic acid-co-vinyl acetate) potassium, poly(ethylene oxide-co-formaldehyde-co-4-nonylphenol) phosphate, poly (maleic anhydride-co-styrene) 2-butoxyethyl ester, ammonium salt, cationic liposomes, anionic liposomes, cationic micelles, anionic micelles, and charged cyclodextrans including sulfobutyl ether P-cyclodextrans.

The concentration range of polyelectrolyte in the electrode can be from about 0.1% to about 99%. A more preferable range is from about 0.25% to about 30%.

For the purpose of illustration and not limitation, another embodiment of the invention relates to an iontophoretic device for carrying out the aforementioned method, the device comprising first and second electrode assemblies and an electrical current source. The electrode assemblies are adapted to be placed in ion transmitting relation with the body tissue. The first electrode assembly comprises the electrode toward which the analyte extracted from the body is driven. The second electrode assembly serves to close the electrical circuit through the body. The circuit is completed by the electrical current source.

If the analyte to be extracted is positively charged or uncharged, then the first electrode assembly will comprise the negatively charged electrode (the cathode) and the second electrode assembly will comprise the positively charged electrode (the anode). If the analyte to be extracted from the body is negatively charged, then the first electrode assembly will comprise the positively charged electrode (the anode) and the second electrode assembly will comprise the negatively charged electrode (the cathode).

Suitable electrode assemblies are well known in the art and any conventional iontophoretic electrode assembly may be used. Suitable electrodes are, for example, disclosed in U.S. Pat. Nos. 4,744,787 to Phipps et al., 4,752,285 to Petelenz et al., 4,820,263 to Spevak et al., 4,886,489 to Jacobsen et al., 4,973,303 to Johnson et al., and 5,125,894 to Phipps et al.

The electrical current may be applied as direct current (DC), alternating current (AC), pulsed DC current, or any combination thereof. Pulsed DC methods are discussed, for example, in U.S. Pat. No. 5,019,034 to Weaver et al. and U.S. Pat. No. 5,391,195 to Van Groningen. Combination pulsed direct current and continuous electric fields are discussed, for example, in U.S. Pat. No. 5,968,006 to Hofmann. U.S. Pat. Nos. 5,135,478 and 5,328,452 to Sabalis, for example, discuss iontophoretic methods that include generating a plurality of waveforms that can be separate or overlapping and that can include an AC signal. U.S. Pat. No. 5,421,817 to Liss et al. discusses the use of a complex set of overlapping waveforms that includes a carrier frequency and various modulating frequencies that collectively are said to enhance delivery. Co-pending applications “METHODS FOR DELIVERING AGENTS USING ALTERNATING CURRENT” by Li et al., Attorney Docket No. 16014-000200US filed Feb. 18, 2001 and “METHODS FOR EXTRACTING SUBSTANCES USING ALTERNATING CURRENT” by Li et al., Attorney Docket No. 16014-000300US filed Feb. 18, 2001, disclose suitable methods of applying AC current alone or in conjunction with a DC prepulse or concomitant DC offset.

The polyelectrolyte or composite of polyelectrolytes will be contained in a reservoir connected to the electrode of the first electrode assembly. Suitable reservoir-containing electrode assemblies are disclosed in, for example, U.S. Pat. No. 4,702,732 to Powers et al., U.S. Pat. No. 5,302,172 to Sage, Jr. et al. and U.S. Pat. No. 5,328,455 to Lloyd et al. and will be well known to those skilled in the art. Examples of such reservoirs or sources include a pouch as described in U.S. Pat. No. 4,250,878 to Jacobsen, a pre-formed gel body as disclosed in U.S. Pat. No. 4,382,529 to Webster and U.S. Pat. No. 4,474,570 to Ariura, et al., a receptacle containing a liquid solution as disclosed in U.S. Pat. No. 4,722,726 to Sanderson et al, a wetable woven or non-woven fabric, a sponge material, or any combination thereof.

It will be appreciated by those working in the field that the methods disclosed herein can be used in the extraction of a wide range of substances. The methods can generally be utilized to extract any substance or mixture of substances that is in a system (e.g., circulatory system) of the subject and that can be transported across a body surface. When the tissue is human skin, the substance or substances are either endogenous or otherwise introduced into the body by some means. Thus, the substance or substances can be molecules that are markers of disease states, pharmaceutical agents administered to the subject, substances of abuse, ethanol, electrolytes, minerals, hormones, peptides, metal ions, nucleic acids, genes, and enzymes, or any metabolites, conjugates, or other derivatives of the aforementioned products. In some instances, more than one substance can be extracted and monitored simultaneously. In yet other instances, similar or differing substances can be extracted at each electrode, with each electrode containing a similar or different polyelectrolyte.

Substances that can be monitored further include, but are not limited to, oligosaccharides, monosaccharides (e.g., glucose), various organic acids (e.g., pyruvic acid and lactic acid), alcohols, fatty acids, cholesterol and cholesterol-based compounds, and amino acids. A number of different substances that correlate with particular diseases or disease states can be monitored. For example, phenylalanine levels can be ascertained to assess treatment of phenylketonuria, which is manifested by elevated blood phenylalanine levels. Examples of metals that can be monitored include, but are not limited to, zinc, iron, copper, magnesium, and potassium.

The methods can be utilized to assess the concentration of various pharmacologically active agents that have been administered for either therapeutic or prophylactic treatment. Examples of such substances include, but are not limited to, analeptic agents; analgesic agents; anesthetic agents; antiasthmatic agents; antiarthritic agents; anticancer agents; anticholinergic agents; anticonvulsant agents; antidepressant agents; antidiabetic agents; antidiarrheal agents; antiemetic agents; antihelminthic agents; antihistamines; antihyperlipidemic agents; antihypertensive agents; anti-infective agents; antiinflammatory agents; antimigraine agents; antineoplastic agents; antiparkinsonism drugs; antipruritic agents; antipsychotic agents; antipyretic agents; antispasmodic agents; antitubercular agents; antiulcer agents; antiviral agents; anxiolytic agents; appetite suppressants; attention deficit disorder and attention deficit hyperactivity disorder drugs; cardiovascular agents including calcium channel blockers, antianginal agents, central nervous system (“CNS”) agents, beta-blockers and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide drugs; psychostimulants; sedatives; steroids; smoking cessation agents; sympathomimetics; tranquilizers; vasodilators; β-agonists; and tocolytic agents; or active metabolites thereof.

Examples of suitable background ions include, but are not limited to, polystyrene sulfonate; poly-N-acetylglucosamine; polyadenylic acid; polyadenylic acid-deca-thymidylic acid; polyadenylic acid-dodeca-thymidylic acid; polyadenylic-cytidylic acid; polyadenylic-cytidylic-guanylic acid; polyadenylic-cytidylic-uridylic acid; polyadenylic-guanylic acid; polyadenylic-guanylic-uridylic acid; polyadenylic-polyuridylic acid; polyadenylic-uridylic acid; polyanetholesulfonic acid; polyanhydrogalacturonic acid; poly-L-arginine; poly-L-asparagine; polybenzylamine acid; polybrene; poly-CBZ-amino acids; polycytidylic acid; polycytidylic inosinic acid; polydeoxyadenylic acid; polydeosyadenylic acid-polythymidylic acid; poly(deoxyadenylic-deoxy-cyticylic)-poly(deoxy-guanylic-thymidylic) acid; polydeoxyadenylic-thymidylic acid; polydeoxycytidylic acid; polydeoxycytidylic-thymidylic acid; polydeoxyguanylic-deoxycytidylic acid; polydeoxyguanylic-polydeoxycytidylic acid; polydeoxyinosinic-deoxycytidylic acid; polydeoxythymidylic acid; polygalacturonic acid; polyglutamic acid; polyguanylic acid; polyguanylic-uridylic acid; polyinosinic acid; polyinosinic-polycytidylic acid; polyinosinic-uridylic acid; polyoxyethylene bis(acetic acid); polythymidylic acid; polyuridylic acid; polyvinyl chloride; polyvinyl sulfate; poly-(α,β)-DL-aspartic acid; poly-L aspartic acid; poly-L-glutamic acid; trisodium timetaphosphate; hexa-ammonium tetrapolyphosphate; pentasodium tripolyphosphate; polyphosphoric acid; dicalcium pyrophosphate; ferric pyrophosphate; tetrapotassium pyrophosphate; disodium pyrophosphate; dextran sulfate; cyclodextran sulfates; or salts or derivatives thereof.

It is to be understood that while the invention has been described in conjunction with the preferred specific embodiments thereof, the foregoing description, as well as the examples that follow, are intended to illustrate and not limit the scope of the invention. Other aspects, advantages, and modifications will be apparent to those skilled in the art to which the invention pertains. All patents, patent applications, journal articles, and other references cited herein are incorporated by reference in their entireties.

### EXAMPLES

Materials:

Conductive silver paint was purchased from Ladd Research Technologies (Williston, Vt.) and silver foil from EM-Science (Gibbstown, N.J.). Silver chloride powder, phosphate buffered saline (PBS, pH 7.4) tablets, agarose, and dextran sulfate (average molecular weight 500,000) were purchased from Sigma (St. Louis, Mo.). Polystyrene sulfonate standards (1,300 and 18,000 with a narrow polydipsersity with a M_{w}/Mₙ of 1.2) were purchased from Polysciences, Inc., (Warrington, Pa.) and ¹⁴C-Mannitol was purchased from American Radiochemical Corp (St. Louis, Mo.). Ultimate Gold® scintillation cocktail was purchased from Packard (Meriden, Conn.) and liquid scintillation counting was performed by a Packard TriCarb Model 1900 TR liquid scintillation analyzer. A Phoresor-II PM 700 (Iomed, Inc., Salt Lake City, Utah) was used as the iontophoretic power supply. Human epidermal membrane was obtained from licensed sources and experiments were conducted under local IRB approval.

Methods:

All of the following experiments were conducted using a side-by-side type diffusion cell with an open diffusional area of 0.85 cm². The cells were separated by a piece of dermatomed, heat-separated human epidermal membrane with the stratum corneum facing the receiver compartment. Each side of the diffusion cell had a 2 ml volume and was stirred at 350 rpm with a magnetic stir bar.

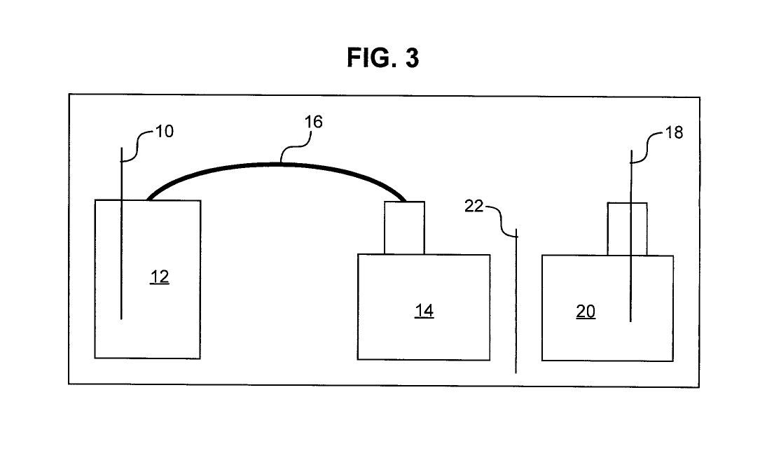

The receiver compartment was filled with either PBS or the electroosmotic-enhancing agent. In each experiment, the donor compartment contained PBS spiked with 30 μl ¹⁴C-mannitol/ml. The cathode was prepared by dipping a silver foil strip into a 1:1 (w/w) mixture of conductive silver paint and finely ground silver chloride. The anode was a piece of silver foil dipped in the conductive silver paint alone. After dipping, the electrodes were hung and allowed to cure at room temperature overnight. The system setup is illustrated in FIG. 3. The negatively charged cathode 10 was placed into a reservoir 12 containing either phosphate buffered saline, pH 7.4. The reservoir 12 was connected to the receiver chamber 14 with a salt bridge 16 containing 2% agarose and the electroosmotic enhancing agent or PBS. The salt bridge 16 was necessary to impede the transport of Cl⁻ into the receiver chamber 14 that was electrochemically liberated from the cathode 10 by the passage of the electrical current. The positively charged anode 18 was placed in the donor compartment 20. A human epidermal membrane 22, as discussed above, separated the donor compartment 20 and the receiver chamber 14. A current of 0.1 mA was passed between the two electrodes during the experiment.

Each experiment was run for 3 consecutive days. On day 1, the experiment was conducted with PBS in the donor chamber, the salt bridge, the reservoir, and the receiver chamber. On day 2, the PBS in the reservoir, salt bridge, and receiver chamber was replaced with the electroosmotic-enhancing agent. This allowed each piece of membrane to serve as its own control. Day 3 again saw PBS in both electrode chambers and served as a control to ensure that the polyelectrolyte did not evince its enhancement through irreversible perturbation of the membrane. In all cases, the permeability from day 3 was not statistically different than day 1. The day 3 results have, therefore, been omitted for clarity.

Every 45 minutes during the experimental run, 100 μl of the receiver solution was withdrawn and mixed with 10 ml of scintillation cocktail. Permeability was calculated from the cumulative dpm vs. time plot. All experiments were run in at least triplicate.

### Results

The results from the above-described experimental examples are presented in Tables 1 and 2 below.

TABLE 1
Measurement of mannitol electroosmotic enhancement between
PBS as the extraction medium and the electroosmotic-enhancing
polyelectrolyte agent as the extraction medium during the first
2¼ hours. The normalized cumulative amount is the cumulative
DPM at 135 minutes in the receiver chamber divided by the DPM
initially present in the donor chamber. PSS = polystyrene sulfonate.

| | | | Mean | |
|---|---|---|---|---|
| | | Mean | Normalized | |
| | | Normalized | Cumulative | |
| | Enhancing | Cumulative | Amount with | Enhance- |
| Exp | Agent/Concentration | Amount in | Enhancing | ment |
| # | (% w/v) | PBS | Agent (cm/s) | Factor |
| 1 | PSS 1,300/13% | 0.012 | 0.113 | 9.4 |
| 2 | PSS 18,000/13% | 0.036 | 1.033 | 28.7 |
| 3 | PSS 18,000/2% | 0.044 | 0.125 | 2.9 |
| 4 | Dextran Sulfate/1.67% | 0.039 | 0.146 | 3.8 |
| 5 | Dextran Sulfate/0.8% | 0.024 | 0.136 | 5.6 |

TABLE 2
Intersample variability for mannitol flux as measured by the
standard error of the mean (SEM) of the steady state
permeability. The standard error of the mean is the standard
deviation normalized for the mean ((Standard Deviation/
Mean)*100%)). N = 3 for each experiment.

| | | Mean Steady | Mean Steady State |
|---|---|---|---|
| | Enhancing | State PBS | Permeability with |
| | Agent/Concentration | Permeability | Enhancing Agent |
| Exp # | (% w/v) | SEM | SEM |
| 1 | PSS 1,300/13% | 81.5% | 37.2% |
| 2 | PSS 18,000/13% | 62.3% | 33.1% |
| 3 | PSS 18,000/2% | 66.8% | 45.1% |
| 4 | Dextran Sulfate/1.67% | 29.6% | 64.5% |
| 5 | Dextran Sulfate/0.8% | 55.9% | 16.2% |

From Table 1 above, it is evident that when chloride ions are replaced by large polyelectrolyte ions in the receiver compartment, the electroosmotic flux of mannitol towards the receiver chamber substantially increases, with the average enhancement ranging from almost 3 to 29 fold. From this example, it is clear that the present invention provides an important advantage over the prior art of Santi and Guy, clearly improving over their two-fold flux enhancement in every case studied.

In addition, with the exception of 1.67% dextran sulfate, Table-2 demonstrates that replacement of chloride with a large polyelectrolyte substantially reduces the inter-sample variability as measured by the standard error of the mean. The replacement of the highly mobile chloride ion by the relatively immobile polyelectrolyte improves the variability in the permeability observed between subjects, often by two-fold or more.

We claim:

1. An iontophoresis device that increases analyte flux during reverse iontophoresis conducted on a region of body tissue comprising:
a) a first electrode assembly adapted to be placed in ion conducting and analyte receiving relation with the body tissue comprising:
(i) a reservoir for collecting and containing an analyte extracted from the body; and
(ii) a first polyelectrolyte composition;
b) a second electrode assembly adapted to be placed in ion transmitting relation with the body tissue at a location spaced apart from the first electrode assembly; and
c) an electrical current source, electrically connected to the first and second electrode assemblies.

2. The device of claim 1, wherein the body tissue is skin.

3. The device of claim 1, wherein the body tissue is mucosal tissue.

4. The device of claim 1, wherein the electrical current is direct current.

5. The device of claim 1, wherein the electrical current is alternating current.

6. The device of claim 1, wherein the electrical current comprises both alternating and directed current superimposed over each other.

7. The device of claim 1, wherein the second electrode assembly contains a similar polyelectrolyte composition.

8. The device of claim 7, wherein the second polyelectrolyte composition is identical to the first polyelectrolyte.

9. The device of claim 7, wherein the second polyelectrolyte is different than the first polyelectrolyte.

10. The device of claim 1, wherein said region of body tissue has an area in the range of less than approximately 1 cm² to greater than 100 cm².

11. The device of claim 10, wherein the said region of body tissue has an area in the range of 5 cm² to 30 cm².

12. The device of claim 1, wherein the device is suitable for analyte extraction that is carried out for a time period in the range of less than 10 minutes to greater than 24 hours.

13. The device of claim 12, wherein the device is suitable for analyte extraction that is carried out for a time period in the range of about 1 hour to 12 hours

14. The device of claim 12, wherein the device is suitable for analyte extraction that is carried out for a time period in the range of about 12 hours to 24 hours.

15. The device of claim 1, wherein the extracted analyte is glucose.

16. The device of claim 1, wherein the extracted analyte is phenylalanine.

17. The device of claim 1, wherein the extracted analyte is a marker of a disease state, a pharmaceutical agent administered to the subject, a substance of abuse, ethanol, an electrolyte, a mineral, a hormone, a peptide, a metal ion, a nucleic acid, a gene, an enzyme, or any metabolite, conjugate, or other derivative of the aforementioned products.

18. The device of claim 1, wherein the extracted analyte is an oligosaccharide, monosaccharide, organic acid, alcohol, fatty acid, cholesterol, cholesterol-based compound, amino acid, zinc, iron, copper, magnesium, or potassium.

19. The device of claim 1, wherein the extracted analyte is a pharmacologically active agent that has been administered for either therapeutic or prophylactic treatment, including analeptic agents; analgesic agents; anesthetic agents; antiasthmatic agents; antiarthritic agents; anticancer agents; anticholinergic agents; anticonvulsant agents; antidepressant agents; antidiabetic agents; antidiarrheal agents; antiemetic agents; antihelminthic agents; antihistamines; antihyperlipidemic agents; antihypertensive agents; anti-infective agents; antiinflammatory agents; antimigraine agents; antineoplastic agents; antiparkinsonism drugs; antipruritic agents; antipsychotic agents; antipyretic agents; antispasmodic agents; antitubercular agents; antiulcer agents; antiviral agents; anxiolytic agents; appetite suppressants; attention deficit disorder and attention deficit hyperactivity disorder drugs; cardiovascular agents including calcium channel blockers, antianginal agents, central nervous system (“CNS”) agents, beta-blockers, and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide drugs; psychostimulants; sedatives; steroids; smoking cessation agents; sympathomimetics; tranquilizers; vasodilators; β-agonists; and tocolytic agents; or metabolites thereof.

20. The device of claim 1, wherein the one or more analytes are extracted concomitantly.

21. The device of claim 1, wherein one or more analytes are extracted concomitantly at the second electrode.

22. A method for extracting an analyte from a region of body tissue, comprising:
(a) placing in contact with the body tissue a first electrode assembly comprising an electrically conducting medium comprising a first polyelectrolyte composition that cannot be readily transported into and through the body tissue when an electrical current is applied;
(b) placing in contact with the body tissue a second electrode assembly adapted to be placed in ion transmitting relation with the body surface at a location spaced apart from the first electrode assembly; and
(c) applying an electrical current across the region of body tissue via the first and second electrode assemblies, with a voltage and duration effective to induce electroosmosis and transport the analyte to the first electrode assembly at a.

23. The method of claim 22, wherein the tissue is skin.

24. The method of claim 22, wherein the tissue is mucosal tissue.

25. The method of claim 22, wherein the electrical current is applied as a direct current.

26. The method of claim 22, wherein the electrical current is applied as an alternating current.

27. The method of claim 22, wherein the electrical current is applied as both alternating and direct current superimposed over one another.

28. The method of claim 22, wherein the second electrode assembly contains a second polyelectrolyte composition.

29. The method of claim 28, wherein the polyelectrolyte composition is identical to the first polyelectrolyte composition.

30. The method of claim 28, wherein the polyelectrolyte composition is different than the first polyelectrolyte composition.

31. The method of claim 22, wherein the extracted analyte is glucose.

32. The method of claim 22, wherein the extracted analyte is phenylalanine.

33. The method of claim 22, wherein the extracted analyte is a marker of a disease state, a pharmaceutical agent administered to the subject, a substances of abuse, ethanol, an electrolyte, a mineral, a hormone, a peptide, a metal ion, a nucleic acid, a gene, an enzyme, or any metabolites, conjugates, or other derivatives of the aforementioned products.

34. The method of claim 22, wherein the extracted analyte is an oligosaccharide, monosaccharide, organic acid, alcohol, fatty acid, cholesterol, cholesterol-based compound, amino acid, zinc, iron, copper, magnesium, or potassium.

35. The method of claim 22, wherein the extracted analyte is a pharmacologically active agent that has been administered for either therapeutic or prophylactic treatment, including analeptic agents; analgesic agents; anesthetic agents; antiasthmatic agents; antiarthritic agents; anticancer agents; anticholinergic agents; anticonvulsant agents; antidepressant agents; antidiabetic agents; antidiarrheal agents; antiemetic agents; antihelminthic agents; antihistamines; antihyperlipidemic agents; antihypertensive agents; anti-infective agents; antiinflammatory agents; antimigraine agents; antineoplastic agents; antiparkinsonism drugs; antipruritic agents; antipsychotic agents; antipyretic agents; antispasmodic agents; antitubercular agents; antiulcer agents; antiviral agents; anxiolytic agents; appetite suppressants; attention deficit disorder and attention deficit hyperactivity disorder drugs; cardiovascular agents including calcium channel blockers, antianginal agents, central nervous system (“CNS”) agents, beta-blockers, and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide drugs; psychostimulants; sedatives; steroids; smoking cessation agents; sympathomimetics; tranquilizers; vasodilators; β-agonists; and tocolytic agents; or metabolites thereof.

36. The method of claim 22, wherein the one or more analyte is extracted concomitantly.

37. The method of claim 22, wherein the first electrode assembly comprises a reservoir electrode containing the polyelectrolyte and adapted to be placed in analyte receiving relation with a body tissue and having a receptacle to collect the extracted analyte.

38. The method of claim 22, wherein said region of body tissue has an area in the range of less than approximately 1 cm² to greater than 100 cm².

39. The method of claim 38, wherein the said region of body tissue has an area in the range of 5 cm² to 30 cm².

40. The method of claim 22, wherein the analyte extraction is carried out for a time period in the range of less than 10 minutes to greater than 72 hours.

41. The method of claim 40, wherein the analyte extraction is carried out for a time period in the range of about 1 hour to 12 hours.

42. The method of claim 40, wherein the analyte extraction is carried out for a time period in the range of about 12 hours to 24 hours.

43. An improved method for extracting an analyte from a region of body tissue, comprising:
(a) placing a first electrode assembly and a second electrode assembly on an individual's body surface in ion-transmitting relation thereto, the first and second electrode assemblies spaces apart at a selected distance, and
(b) applying an electrical current across the region of body tissue via the first and second electrode assemblies, with a voltage and duration effective to induce electroosmosis and transport the analyte to the first electrode assembly at a transport rate having a mean steady state permeability that varies when the method is applied to different regions of body tissue,
the improvement comprising incorporating a polyelectrolyte composition into the first electrode assembly that exhibits significantly impeded transport into the body tissue when an electrical current is applied, said polyelectrolyte composition effective to provide a substantial decrease in the variability the mean steady state permeability when the method is applied to different regions of body tissue.

44. The method of claim 43, wherein the decrease in the variability of the mean steady state permeability is from about 5% to about 95% relative to the mean steady state permeability variability observed without the incorporation of the polyelectrolyte composition.

45. The method of claim 44, wherein the decrease in the variability of the mean steady state permeability is from about 10% to about 80% relative to the mean steady state permeability variability observed without the incorporation of the polyelectrolyte composition.

46. The method of claim 45, wherein the decrease in the variability of the mean steady state permeability is from about 20% to about 70% relative to the mean steady state permeability variability observed without the incorporation of the polyelectrolyte composition.
